(19) United States
(12) Patent Application Publication
(10) Pub. No.: US 2012/0034312 A1
(43) Pub. Date: Feb. 9, 2012

(54) 4-ISOPROPYL-3-METHYLPHENOL FOR THE TREATMENT OF INFLAMMATION

(75) Inventor: Jingjun Yang, Surrey (GB)

(73) Assignee: Glaxo Group Limited

(21) Appl. No.: 13/262,646

(22) PCT Filed: Apr. 1, 2010
(86) PCT No.: PCT/EP2010/054394

§ 371 Date: Oct. 3, 2011

(30) Foreign Application Priority Data
Apr. 3, 2009 (GB) 0905864.5

(51) Int. Cl. A61K 33/42; A61K 33/30; A61P 29/00; A61K 31/60; A61K 31/05; C07C 39/06; A61K 31/315

(52) U.S. Cl. 424/604; 568/781; 424/641; 514/494; 514/164; 514/731

(57) ABSTRACT

This invention relates to the use of 4-isopropyl-3-methylphenol (IPMP) for combating inflammation and compositions comprising IPMP for such use.

This invention relates to the use of 4-isopropyl-3-methylphenol (IPMP) for combating (ie helping to prevent, inhibit or treat) inflammation, and compositions comprising IPMP for such use. Suitable compositions include pharmaceutical compositions and personal care compositions for oral, throat and skin care, in particular oral care compositions.

Inflammation of the oral mucosa in the oral cavity can be an issue for people with either natural teeth or denture wearers. Inflammation can manifest in a number of ways, for example as swollen, sore, painful and bleeding gums and/or oral ulcers and blisters. Inflammation may lead to gingivitis and if left untreated will lead to periodontitis with possible loss of the alveolar bone in the periodontium. Inflammation can also occur due to poor hygiene in the mouth.

Anti-inflammatory agents, both steroidal and non-steroidal, are well known in the art. Examples include aspirin, indomethacin, corticosteroids and ibuprofen. All of these agents claim to combat inflammation in humans. Unfortunately, these agents are also known to cause side-effects, such as gastrointestinal disorders. Furthermore, these agents are not known for incorporation into oral care products since the traditional performance metric of these products has been gum health or cleaning efficacy instead of therapeutic efficacy.

IPMP is an ingredient with an established safety profile, is known for its antimicrobial properties and is acceptable for oral use from a toxicological and regulatory perspective.

U.S. 2008/0253976 (Procter & Gamble) describes personal care compositions for oral, throat and skin care comprising a blend of a first component selected from citral, neral, geranial, geraniol and nerol and a second component selected from eucalyptol, eugenol and carvenol, which blend is described to exhibit both antibacterial and anti-inflammatory activities, stated to be particularly effective against bacteria-mediated inflammatory diseases such as gingivitis. Optionally the blend may further comprise additional antimicrobial and/or anti-inflammatory components including amongst many other potential agents, IPMP.

US 2007/0053849 (Procter & Gamble) describes topical oral care compositions comprising the combination of an anti-inflammatory agent with an antibacterial agent. Examples of anti-inflammatory agents include vitamin compounds; curcuminoids; oils and extracts from spices and botanicals; oils and extracts from thyme, oregano and sage; neem oil; flavonoids and flavones; and phenolics from plant sources. Examples of antibacterial agents include cetyl pyridinium chloride, stannous ion agent, zinc ion agent, copper ion agent, iron ion agent, triclosan, ascorbyl stearate, oleoyl sarcosine, dioctyl sulfosuccinate, alkyl sulphate and mixtures thereof. The use of IPMP is not described.

The present invention is based upon the discovery that IPMP has intrinsic anti-inflammatory activity. In particular it has been found that IPMP inhibits inflammation as measured by cellular release of the inflammatory cytokine marker, prostaglandin E2 (PGE2), when used in a therapeutically effective amount. In a clinical setting, PGE2 is associated with the manifestations of inflammation such as pain, swelling, redness and heat associated with irritation or injury to body tissues. See for example P. Davies, P. J. Bailey, M. M. Goldenberg and A. W. Ford-Hutchinson, “The role of arachidonic acid oxygenation products in pain and inflammation”, Annu. Rev. Immunol. 2 (1984), pp. 335-357.

It has also been found that IPMP, when used in combination with the known anti-inflammatory agent zinc provides enhanced anti-inflammatory efficacy. The anti-inflammatory activity of zinc is described, for example in “Zinc: mechanisms of host defense”. The Journal of Nutrition, (2007) May;137(5):1345-9.

Accordingly, in a first aspect, the present invention provides a composition for use in combating inflammation which composition comprises IPMP.

In a second aspect, the present invention provides the use of IPMP in the manufacture of a composition for combating inflammation.

In a third aspect, the present invention provides a method for combating inflammation in a patient in need thereof, said method comprising administering a composition comprising an effective amount of IPMP.

The term “inflammation” as used herein at all occurrences refers to the response of body tissues to injury or irritation characterized by pain and swelling and redness and heat.

Suitably the IPMP is present in an amount from 0.01% to 1.00%, for example from 0.04% to 0.20% or 0.05% to 0.10% by weight of the total composition.

Suitably the composition of the present invention further comprises a source of zinc ions.

Suitably the source of zinc ions, as defined as the zinc portion of a corresponding salt, is present in an amount from 0.01% to 2.50%, for example from 0.04% to 0.70% by weight of the total composition.

Suitably the source of zinc ions is a zinc salt such as zinc chloride, zinc citrate, zinc acetate, zinc sulphate, zinc gluconate, zinc salicylate, zinc lactate, zinc maleate, zinc malate, zinc tartrate, zinc carbonate, zinc phosphate, zinc oxide or zinc sulphate. Additional zinc salts are described in U.S. Pat. No. 4,022,880 (Vinson et al).

A preferred zinc salt is zinc chloride.

In one embodiment the composition of the present invention is a pharmaceutical composition comprising a pharmaceutically acceptable carrier or excipient.

Suitable pharmaceutical dosage forms for oral administration include tablets and capsules. Suitable pharmaceutical dosage forms for topical administration include creams and ointments which can be applied to the skin.

Examples of pharmaceutically acceptable carriers or excipients are described in the Handbook of Pharmaceutical Excipients (eg the Fourth Edition, 2003, published by the Pharmaceutial Press).

In another embodiment the composition of the present invention is a personal care composition for oral, throat or skin care, comprising a carrier or excipient acceptable for personal care use. Examples of suitable personal care dosage forms and carriers or excipients are described in U.S. 2008/0253976 (Procter & Gamble), the contents of which are herein incorporated by reference.

In a preferred embodiment the composition of the present invention is an oral care composition comprising an orally acceptable carrier or excipient.

Oral care compositions of the present invention are typically formulated in the form of toothpastes, sprays, mouthwashes, gels, lozenges, chewing gums, tablets, pastilles, instant powders, oral strips, buccal patches, wound dressings and denture adhesives.

Oral care compositions of the present invention may comprise one or more active agents conventionally used in oral healthcare compositions, for example, a fluoride source, a desensitising agent, an anti-plaque agent; an anti-calculus agent, a whitening agent, an oral malodour agent or a mixture of at least two thereof. Such agents may be included at levels to provide the desired therapeutic effect. The oral care composition may further comprise an additional anti-inflammatory agent, an anti-oxidant, anti-fungal or wound healing agent.

Suitable anti-inflammatory agents include vitamins (vitamin E, vitamin B2, folic acid, etc.); NSAIDS (aspirin, ibuprofen, ketoprofen, etc.); steroidal anti-inflammatory compounds (corticosteroids); natural extracts (tumeric, green tea extract, ginger extract, etc.); biological compounds (omega-3 fatty acids, ethyl pyruvate, etc).

Suitable anti-oxidant agents include vitamins (vitamin A, vitamin C, vitamin E, etc.); biological compounds (resveratrol, EGCG, lycopene, etc.); food preservatives (TBHQ, BHA, BHT, parabens, etc); and natural extracts (soy, grape, olive oil, etc).

Oral care compositions of the present invention will contain additional formulating agents such as abrasives, thickening agents, surfactants, humectants, flavouring agents, sweetening agents, opacifying or colouring agents, preservatives and water, selected from those conventionally used in the oral hygiene composition art for such purposes.

Suitable oral care actives and orally acceptable carriers or excipients (ie the above-noted formulating agents) are described for example in US 2007/0053849 (Procter & Gamble).

Suitably the oral care composition comprises an anionic surfactant which has been found to enhance the antimicrobial efficacy of the IPMP and/or source of zinc ions in the oral care composition.

Suitable examples of anionic surfactants include alkali metal C₈₋₁₈alkylsulphates (eg sodium lauryl sulphate, SLS), alkali metal C₈₋₁₈alkylarylsulphonates (eg sodium dodecylbenzene sulphonate, SDDBS), alkali metal sulphonated monoglycerides of C₁₀₋₁₈ alkyl fatty acids (eg sodium coconut monoglyceride sulphonate), alkali metal C₁₀₋₁₅alkyl sulphoacetates (eg sodium lauryl sulphoacetate), and alkali metal salts of sarcosinates, isethionates and taurates, such as sodium lauryl sarcosinate, sodium lauroyl sarcosinate, sodium myristoyl sarcosinate, sodium palmitoyl sarcosinate, sodium stearoyl sarcosinate, sodium oleoyl sarcosinate and sodium lauroyl isethionate.

Suitably the anionic surfactant is an alkali metal C₈₋₁₈alkylsulphate or an alkali metal C₈₋₁₈ alkylarylsulphonate or an alkali metal sarcosinate or a mixture thereof.

Most suitable anionic surfactants for use in the present invention are SDDBS, SLS, sodium lauryl sarcosinate and mixtures thereof, preferably in total concentration of 0.1% to 2.5%, more preferably 0.5% to 2.0%, even more preferably 1.0% to 1.5%

The compositions according to the present invention may be prepared by admixing the ingredients in the appropriate relative amounts in any order that is convenient and if necessary adjusting the pH to give a final desired value.

When the composition is in the form of a toothpaste, it is suitable for containing in and dispensing from a laminate tube or a pump as conventionally used in the art.

The invention will now be described by way of the following non-limiting examples.

### EXAMPLE 1

Anti-inflammatory activity was assessed on classical inflammation model LPS stimulated Raw 264.7 Macrophage cells. Inflammation response was quantified by measurements of the release of Prostaglandin E2 (PGE₂) from the cells. Cells were exposed to E.coli lipopolysaccharide (LPS) with or without IPMP for 24 hours prior to assay. IPMP significantly reduced the level of general inflammation induced by the exposure of the cells to LPS. IPMP by itself did not irritate the cells. All values were normalized to cell viability of same sample, then to the inflammatory response of the cells without the addition of agents or LPS (100%).

| | PGE₂ Response | PGE₂ Response |
|---|---|---|
| Treatment | (without LPS) | (with LPS) |
| Media only | 100 ± 6.0 | 412 ± 18.0 |
| Media with 50 μM IPMP | 101 ± 0.0 | 141 ± 6.4 |

The results indicate that IPMP at a nonirritative dose, ie a dose of IPMP (50 μM in this case) that will neither cause cell death nor affect cell proliferation, provides strong anti-inflammatory activity.

### EXAMPLE 2

To further evaluate anti-inflammatory activity of IPMP in the oral cavity, a similar experiment was conducted on Human Gingival Fibroblasts. Cells were exposed to LPS as well as three non-irritating doses of IPMP (see example 1).

| | PGE₂ Response | PGE₂ Response |
|---|---|---|
| Treatment | (without LPS) | (with LPS) |
| Media only | 100 ± 6.0 | 164 ± 9.1 |
| Media with 50 μM IPMP | — | 103 ± 13.3 |
| Media with 10 μM IPMP | — | 127 ± 21.6 |
| Media with 2 μM IPMP | — | 146 ± 46.6 |

These results show that on human oral cells, IPMP suppresses LPS induced inflammation in a dose-dependent manner.

### EXAMPLE 3

Another further experiment with IPMP and zinc chloride was conducted on Human Gingival Fibroblasts.

| | PGE₂ | PGE₂ |
|---|---|---|
| | Response | Response |
| Treatment | (without LPS) | (with LPS) |
| Media only | 100 ± 6.0 | 164 ± 9.1 |
| Media with 10 μM IPMP | — | 127 ± 21.6 |
| Media with 55 μM Zinc Chloride | — | 171 ± 2.9 |
| Media with 1 μM IPMP and | — | 60 ± 10.0 |
| 55 μM of Zinc Chloride | | |

The results show a synergistic anti-inflammatory activity of IPMP and Zinc Chloride (at molar ratio of 1:5.5) on a Human Gingival Fibroblasts model.

EXAMPLES 4-7

| Dentifrice Composition | Ex 4 | Ex 5 | Ex 6 | Ex 7 |
|---|---|---|---|---|
| Raw Material | % w/w | % w/w | % w/w | % w/w |
| Sorbitol, Liquid (Non- | 27.65 | 27.65 | 27.65 | 27.65 |
| Crystallising) | | | | |
| Glycerin (98%) | 4.00 | 4.00 | 4.00 | 4.00 |
| Polyethylene Glycol 300 (PEG | 4.00 | 4.00 | 4.00 | 4.00 |
| 6) | | | | |
| Silica, Dental Type (Zeodent | 14.00 | 14.00 | 14.00 | 14.00 |
| 113) | | | | |
| Silica, Dental Type (Zeofree | 9.00 | 9.00 | 9.00 | 9.00 |
| 153B) | | | | |
| Sodium Lauryl Sulphate | 0.75 | 0.75 | 1.50 | 1.50 |
| Sodium | 0.75 | 0.75 | — | — |
| dodecylbenzenesulphonic acid | | | | |
| Xanthan Gum (“xanth”, Keltrol | 0.80 | 0.80 | 0.80 | 0.80 |
| F) | | | | |
| Carrageenan (“carra”, Genuvisco | 0.40 | 0.40 | 0.40 | 0.40 |
| TPH-1) | | | | |
| Saccharin Sodium | 0.30 | 0.30 | 0.30 | 0.30 |
| Sodium Fluoride | 0.24 | 0.243 | 0.20 | 0.10 |
| Zinc Chloride | 0.50 | 0.50 | 0.50 | 0.50 |
| Titanium Dioxide | 1.00 | 1.00 | 1.00 | 1.00 |
| Flavour | 1.50 | 1.50 | 1.50 | 1.50 |
| Sodium citrate tribasic dihydrate | 1.84 | 1.84 | — | 1.84 |
| Isopropylmethyl phenol | 0.05 | 0.10 | 0.05 | 0.1 |
| Citric acid (anhydrous) | — | — | 0.03 | 0.03 |
| Purified Water | ad 100 | ad 100 | ad 100 | ad 100 |

1. A composition comprising 4-isopropyl-3-methylphenol (IPMP) for use in combating inflammation.

2. A composition according to claim 1 further comprising a source of zinc ions.

3. A composition according to claim 2 wherein the source of zinc ions is selected from zinc chloride, zinc citrate, zinc acetate, zinc sulphate, zinc gluconate, zinc salicylate, zinc lactate, zinc malate, zinc maleate, zinc tartrate, zinc carbonate, zinc phosphate, zinc oxide or zinc sulphate

4. A composition according to claim 3 wherein the source of zinc ions is zinc chloride.

5. A composition according to claim 1 any one of claims 1 which is a pharmaceutical composition comprising a pharmaceutically acceptable carrier or excipient.

6. A composition according to claim 1 any one of claims 1 which is a personal care composition for oral, throat or skin care, comprising a carrier or excipient acceptable for personal care use.

7. A composition according to claim 6 which is an oral care composition comprising an orally acceptable carrier or excipient.

8. A composition according to claim 7 which comprises an anionic surfactant.

9. (canceled)

10. A method for combating inflammation in a patient in need thereof, said method comprising administering a composition comprising an effective amount of IPMP.
